(19) United States
(12) Patent Application Publication
(10) Pub. No.: US 2008/0038311 A1
(43) Pub. Date: Feb. 14, 2008

(54) BONE REPLACEMENT MATERIAL

(75) Inventors: SEBASTIAN VOGT, ERFURT (DE); KLAUS-DIETER KUHN, MARBURG-ELNHAUSEN (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS KULZER GMBH, Hanau (DE)

(21) Appl. No.: 11/830,257

(22) Filed: Jul. 30, 2007

(30) Foreign Application Priority Data
Aug. 9, 2006 (DE) 10 2006 037 362.6

(51) Int. Cl. A61F 2/00

(52) U.S. Cl. 424/423

(57) ABSTRACT

A bone replacement material that is, in particular, paste-like and is characterized in that particulate calcium carbonate is suspended in an aqueous solution that contains at least one water-soluble hemostatic agent, whereby the aqueous solution contains an amount of the hemostatic agent at which it is isotonic.

The subject matter of the invention is a bone replacement material. This bone replacement material is to be suitable, in particular, for augmentation of human spongy bone.

Autologous spongy bone continues to be the so-called gold standard for filling bone defects (J. Jerosch, A. Bader, G. Uhr: Knochen curasan Taschenatlas spezial. Thieme Verlag 2002). Autologous spongy bone affords the best results at this time. Obviously, the use of autologous spongy bone is limited with regard to the maximal volume thereof that can be obtained. For this reason, it is obvious to augment autologous spongy bone using suitable bone replacement materials in order to be able to fill more extensive bone defects. It is customary to mix 2 volume parts of spongy bone and one volume part of bone replacement material.

A multitude of granular ceramic materials made of calcium phosphates, such as hydroxy apatite, β-tricalciumphosphate, and α-tricalciumphosphate, are known in the medical products market (J. M. Rueger, W. Linhart, D. Sommerfeldt: Biologische Reaktionen auf Kalziumphosphatkeramik-Implantationen. Tierexperimentelle Ergebnisse [Biological responses to the implantation of calcium phosphate ceramic materials. Results from animal experiments]. Orthopäde 27 (1998) 89-95.) Examples of these include the bone replacement materials, Endobone®, Calcibone®, Biobase®, BETA-BASE®, and Cerasorb®, that are in common use in Germany. An interesting alternative is provided by paste-like bone replacement materials that are based on nanoparticulate hydroxy apatite and are known in the market under the name of Ostim® (EP0664133 A1). In this context, nanoparticulate hydroxy apatite is suspended in water such that injectable pastes are generated. However, a prerequisite for this material is the availability of nanoparticulate hydroxy apatite which needs to be synthesized in a special manufacturing process. This manufacturing process obviously causes the fabrication costs to be higher as compared to a material of which bulk quantities at pharmaceutical grade are commercially available. Also known are pastes that are a combination of nanoparticulate hydroxy apatite and calcium sulfate (EP1301219 A1). As before, these involve the use of nanoparticulate hydroxy apatite that can be manufactured only at high cost though.

WO03028779A1 describes a bone filling material having calcium salt particles, organic binding agent, and cells from the group of stem cells, osteogenic cells, and osteoprogenic cells, as well as a buffer. The mixture can be injected by means of needles.

US20030055512A1 also relates to an injectable paste that contains biologically degradable calcium compounds such as calcium sulfate, hydroxy apatite, and tricalcium phosphate. The setting time in an aqueous system can be adjusted.

According to EP1243257A1, an amphiphilic organic sulfate, sulfamate, sulfonate, disulfate, disulfonate or trisulfonate is incorporated into an aminoglycoside, lincosamide, 4-quinolone or tetracycline antibiotic in order to attain delayed release of the agent.

The invention is based on the object to provide a cost-efficient, easy-to-manufacture bone replacement material that can be inserted conveniently into bone cavities having a wide variety of shapes and sizes.

The object is met according to the invention as described herein. The object is met in that the bone replacement material contains calcium carbonate that is suspended in the form of particulate calcium carbonate in an aqueous solution that contains at least one water-soluble hemostatic agent, whereby the aqueous solution contains an amount of the hemostatic agent that renders the aqueous solution isotonic. The osmotic pressure of human blood at 37° C. is 7.5 bar. The aqueous solution and blood are isotonic if the osmolality of the solution is 288 mOsmol. Hereinafter, the term isotonic shall be understood to mean that the osmolality of the aqueous solution at 37° C. is in the range of 250-300 mOsmol.

Following its implantation, the bone replacement material shows, on the one hand, volume stability for a period of several days to weeks, and, on the other hand, the bone replacement material attains within a short time a sufficient shape stability to prevent it from migrating by flowing in the first few days after implantation. The bone replacement material does not cause acidification (lowering of the pH value) in the bone defect during resorption. Moreover, the components of the bone replacement material are also natural components of the human body such that incompatibilities and toxic effects are prevented.

Calcium carbonate is only slightly soluble in carbon dioxide-free water and shows a pronounced buffering effect with respect to acids. It is not cytotoxic and no systemic toxic effects are known either. Calcium carbonate is made up of calcium ions and carbonate ions. Both calcium ions and carbonate ions are natural components of the human body. Calcium ions are present in locations including the inorganic substance of the bone, in carbonate apatite, and in the hard substance of the teeth. Carbonate ions are a component of the carbon dioxide-hydrogencarbonate-carbonate buffer in the blood. This buffer ensures that the pH value of the human blood is constant. In aqueous solution, carbonate ions and protons are in a chemical equilibrium with gaseous carbon dioxide. Carbonate ions are excreted from the human body via the lung in the form of carbon dioxide. Calcium carbonate is only slightly soluble in carbon dioxide-free water. Accordingly, only 14 mg of calcium carbonate dissolve per milliliter of carbon dioxide-free water at 20° C. (Römpp-Lexikon Chemie. Eds.: J. Falbe; M. Regitz, 10th, completely revised edition, volume 1, Thieme Verlag 1996, p. 574). In contrast, in the presence of carbon dioxide dissolved in water, calcium carbonate dissolves releasing calcium ions and hydrogen carbonate ions. A total of 0.85 g of calcium carbonate dissolves per milliliter of carbon dioxide-saturated water at 20° C. (Römpp-Lexikon Chemie. Eds.: J. Falbe; M. Regitz, 10th, completely revised edition, volume 1, Thieme Verlag 1996, p. 574). Despite being only slightly soluble in water, this provides for dissolution of calcium carbonate in the human body since dissolved carbon dioxide is always present in the hard and soft tissue.

Calcium carbonate is economically advantageous as compared to nanoparticulate hydroxy apatite in that relevant technical-scale quantities having pharmaceutical quality are available quite cheaply.

Preferably, the bone replacement material is paste-like. A preferred embodiment of the invention is based, on the one hand, on particulate calcium carbonate, at a calcium carbonate content of preferably 55 to 67 mass percent, forming paste-like mixtures with water that can be injected without any difficulty and, on the other hand, on having a hemostatic agent that is dissolved in the water of the paste initiating blood coagulation after the paste-like bone replacement material contacts the blood. This facilitates the, in particular paste-like, bone replacement material becoming fixed in space by the cross-linked fibrin thus generated and, as a result, migration processes of the bone replacement material are prevented or delayed for a period of several days. In this context, it is essential that the hemostatic agent and the water form an isotonic solution. A paste having a calcium carbonate content of 60 mass percent has proven to be optimal with regard to its injectability.

Modifications of calcium carbonate, such as vaterite, calcite and aragonite, are known that can be synthesized by adjusting corresponding parameters of synthesis such as, for example, the reaction temperature. It is advantageous for the known modifications of calcium carbonates or mixtures of these modifications to possibly be contained in the bone replacement material according to the invention.

It is also advantageous for the calcium carbonate to have a particle size of preferably from 1-50 μm. The use of smaller calcium carbonate particles is also feasible. Their size can be in the nanometer range.

Also useful is the use of one or more substances from the group of calcium chloride, calcium acetate, calcium lactate, gelatin as hemostatic agent. It has become evident that both inorganic and organic calcium salts are preferred as compounds that are effective as hemostatic agents. It is a generally known fact that dissolved calcium ions can accelerate the coagulation of blood. Calcium ions are an essential component at several places in the coagulation cascade. They contribute to the activation of factor VII and factor IX and thus to the formation of prothrombin activator. Moreover, calcium ions are essential in the action of thrombin on fibrinogen leading to the formation of fibrin monomers which in turn form the fibrin network under participation of activated factor XIII.

Platelets (thrombocytes) can easily aggregate on gelatin. This can also initiate the coagulation of blood.

Preferably, in addition to calcium carbonate, magnesium carbonate and/or magnesium oxide can also be contained in the, particularly paste-like, bone replacement material, if applicable. Depending on its source, calcium carbonate almost always contains magnesium carbonate. Magnesium ions, like calcium ions, are a natural component of the human body.

It is also useful for one or more antibiotics or other pharmaceutical agents to be contained in the bone replacement material, if applicable. These agents can be contained therein in dissolved and in non-dissolved, suspended form. Antibiotics from the group of the aminoglycoside antibiotics are preferred due to their chemical stability. Particularly useful in this context are the broad-range antibiotics, gentamicin sulfate and tobramycin sulfate. Besides, it is also feasible to incorporate glycopeptide antibiotics, such as vancomycin, teicoplanin and dalbavancin, in the bone replacement material according to the invention. Moreover, fluoroquinolone antibiotics, such as ofloxacin, levofloxacin and moxifloxacin, can also be contained therein. Provided antibiotics are contained in the bone replacement material according to the invention, the bone replacement material can advantageously be used also for temporary filling of bone cavities that are generated during the surgical repair of osteomyelitic bone areas.

The use of other pharmaceutical agents in the bone replacement material, such as antiphlogistics, corticosteroids, and bone growth factors, is also included in the scope of the invention. Amongst the bone growth factors, rhBMP-2 and rhBMP-7 are particularly preferred.

The invention is illustrated by the following examples without limiting the general scope of the invention.

### EXAMPLE 1

Calcium-L-lactate hydrate (Fluka) was added to and dissolved in 500 ml sterile, pyrogen-free water at 37° C. until the osmolality of the solution was 288 mOsmol as measured using an osmometer. Then, 100 ml of this solution were mixed with 65.0 g calcium carbonate (Fluka). A colorless to light-grey viscous paste was thus produced. 50 g of this paste were placed in a conventional plastic syringe. The paste was easy to dispense from this syringe by actuating the plunger.

### EXAMPLE 2

Calcium acetate hydrate (Fluka) was added to and dissolved in 500 ml sterile, pyrogen-free water at 37° C. until the osmolality of the solution was 288 mOsmol as measured using an osmometer. Then, 100 ml of this solution were mixed with 60.0 g calcium carbonate (Fluka). A colorless to light-grey viscous paste was thus produced. 50 g of this paste were placed in a conventional plastic syringe. The paste was easy to dispense from this syringe by actuating the plunger.

What is claimed is:

1. Bone replacement material comprising particulate calcium carbonate suspended in an aqueous solution that comprises at least one water-soluble hemostatic agent in an amount of the hemostatic agent that renders the aqueous solution isotonic.

2. Bone replacement material according to claim 1, which comprises 55 to 67 mass percent calcium carbonate.

3. Bone replacement material according to claim 1, wherein the calcium carbonate has a particle size of from 1-50 μm.

4. Bone replacement material according to claim 1, wherein the hemostatic agent is one or more compounds selected from the group consisting of calcium chloride, calcium acetate, calcium lactate and gelatin.

5. Bone replacement material according to claim 1, which further comprises magnesium carbonate and/or magnesium oxide in addition to calcium carbonate.

6. Bone replacement material according to claim 1, which further comprises one or more antibiotics and/or other pharmaceutical agents.

7. Bone replacement material according to claim 1, which has a consistency of paste.

8. Method of repairing or replacing bone comprising apply bone replacement material according to claim 1 to an area where it is desired to repair or replace bone.
